(19) United States
(12) Patent Application Publication
(10) Pub. No.: US 2017/0027963 A1
(43) Pub. Date: Feb. 2, 2017

(54) DELIVERY OF NON-STEROIDAL AGENTS TO THE BRAIN VIA THE NASAL TRACT TO TREAT NEUROLOGICAL DISORDERS

(71) Applicants: Patrick CROWLEY, Devon, PA (US); David TEMPLETON, Hertfordshire (GB)

(72) Inventors: Patrick CROWLEY, Devon, PA (US); David TEMPLETON, Hertfordshire (GB); Khuloud AL-JAMAL, Greater London (GB); Luigi Gerard MARTINI, Essex (GB); Philip SMITH, Cape May, NJ (US)

(73) Assignees: Patrick CROWLEY, Devon, PA (US); David TEMPLETON, Hertfordshire, PA (GB)

(21) Appl. No.: 15/302,789

(22) PCT Filed: Apr. 10, 2015
(86) PCT No.: PCT/GB2015/051100

§ 371 Date: Oct. 7, 2016

(30) Foreign Application Priority Data
Apr. 10, 2014 (GB) 1406493.5
Nov. 3, 2014 (GB) 1419591.1

(51) Int. Cl. A61K 31/618; A61K 31/12; A61M 11/04; A61K 9/14; A61K 9/16; A61K 9/00; A61K 31/192; A61K 31/60

(52) U.S. Cl. CPC A61K 31/618; A61K 31/192; A61K 31/12; A61K 31/60; A61K 9/14; A61K 9/1611; A61K 9/0043; A61M 11/042

(57) ABSTRACT

There is described a volatile form of an NSAID, and derivatives thereof, for use in the treatment of a neurological disorder. There is also described a method of treatment of a neurological disorder which comprises administering to a mammal a therapeutically effective amount of a volatile form of an NSAID, or a derivative thereof, in vapour form.

### FIELD OF THE INVENTION

The present invention relates to novel formulations, to novel methods of treatment and novel methods of delivering therapeutically active agents.

More particularly, the invention relates to novel formulations, suitable for the treatment of neurological disorders, to their use and methods for treating neurological disorders. In particular, the present invention provides formulations and methods for the delivery of therapeutically active agents via the olfactory tract, thus bypassing the blood brain barrier and delivering the therapeutically active agent direct to the brain.

### BACKGROUND TO THE INVENTION

The invention provides compositions and methods for the therapeutic treatment of neurological disorders.

Neurological conditions can seriously affect a person's ability to carry out normal daily activities. Such neurological conditions include, for example, pain and neurodegenerative disorders, such as, Multiple Sclerosis, dementia, Alzheimer's disease, Parkinson's disease, stroke, traumatic brain injury and the like.

Among older people, Alzheimer's disease is the most common form of dementia and involves parts of the brain that control thought, memory, and language. Despite intensive research, the causes of Alzheimer's disease are still unknown and there is no known cure. Most commonly Alzheimer's disease begins after the age of 60, with the risk of acquiring the disease increasing with age, although younger people may also suffer from Alzheimer's disease.

According to the Alzheimer's Society there are currently 800,000 people with dementia in the UK, and it is estimated that by 2021 there will be over a million people with dementia in the UK.

In the UK 60,000 deaths a year are directly attributable to dementia and it is estimated that by delaying the onset of dementia by 5 years deaths directly attributable to dementia would be reduced by 30,000 a year.

Furthermore, 80% of people living in care homes have a form of dementia or severe memory problems. The financial cost of dementia to the UK was estimated to be in excess of £23 billion in 2012.

Inflammation and local immune responses in the Central Nervous System and associated nerve tracts are now recognized as a feature of neurological conditions, such as, pain and neurodegenerative conditions that include Multiple Sclerosis, Alzheimer's disease, Parkinson's disease, Stroke and traumatic brain injury. Parkinson's disease is caused by progressive loss of dopamine neurons in the substantia nigra region of the brain. The molecular basis for such losses is unclear and current treatments provide only symptomatic relief. They do not slow or reverse the condition. Furthermore treatment becomes less effective over time.

Alzheimer's disease is also associated with brain-related neurodegeneration, with progressive cognitive decline, functional impairment and loss of independence. The condition places enormous financial and psychological burdens on Caregivers and Healthcare Providers. Worldwide prevalence has been estimated as being in the region of 35 million in 2010, with levels projected to increase as life expectancy increases in the developed world.

The number of deaths caused by Alzheimer's disease is generally thought to be underreported, but a recent study has identified Alzheimer's disease as the potential third-leading cause of death in the USA after heart disease and cancer.

Brain injury is also increasingly being recognized as playing a role in conditions loosely described as “Post-Traumatic-Stress Disease”.

Studies have indicated that chronic use of non-steroidal anti-inflammatory drugs (NSAIDs) appears to reduce the risk of acquiring Alzheimer's disease and/or delay the onset of the disease. In a study involving more than 49,000 US veterans aged 55 and older who developed Alzheimer's disease and nearly 200,000 who had no form of dementia, the researchers looked at more than five years of prescription data from the U.S. Veterans Affairs health care system. They found those who were prescribed ibuprofen for more than five years were 40 percent less likely to develop Alzheimer's disease than those who did not. The longer they used ibuprofen the lower their risk.

There is now evidence that long term use of some NSAID's, taken orally for other conditions, reduces the risk of developing Parkinsonism and Alzheimer's disease, or limit disease progression. Such evidence, along with studies in animals, suggests that the pathophysiology of these conditions may involve inflammatory processes and mediators in various locations of the brain. However, chronic oral administration of very high doses of NSAIDs or long term systemic administration of conventional doses of NSAIDs can lead to side effects such as gastric ulceration, gastrointestinal bleeding, kidney dysfunction, hypertension and cardiovascular effects in the case of COX-2 Inhibitors. The risks associated with such side effects on chronic dosage, when drug is administered orally could outweigh the benefits for treating or slowing progression of brain-related diseases.

Furthermore, the brain is one of the most inaccessible sites for delivery of therapeutic agents. Tight junctions at the blood brain barrier and the presence of various metabolizing enzymes provide formidable barriers to delivery by conventional means, such as oral administration. Furthermore, novel particulate-based delivery systems such as liposomes are too large to traverse the blood-brain barrier. If NSAIDs are to be useful and safe for treating or preventing the development of neurological or brain trauma-related conditions a form of delivery is required that provides an appropriate dose “to the right place” i.e. the brain and does not flood the remainder of the biosystem with potentially harmful levels of drug. In particular, the targeted delivery of the NSAID would permit a lower dosage of NSAID to be administered, thus minimising the undesirable side effects commonly found with high dose administration of NSAIDs.

Many reports, designs and devices exist for intranasal administration of medicaments. However, most focus invariably concerns absorption of drug across the nasal mucosa and passage to the systemic circulation. Products and devices are also available for delivery of volatile materials such as camphor, menthol and methyl salicylate as decongestants. However, any putative effect seems to be “local” i.e. to shrink swollen sinus membranes in the nasal cavity” rather than delivery of such materials to the systemic or central nervous system. Substance abuse practices include intranasal ingestion of pharmaceutically active agents, such as cocaine, e.g. by “snorting” (slang). Treatments such as aromatherapy are also based on the inhalation of volatile agents.

However, there is no validated prior art that teaches a mode of and target for drug delivery to the Central Nervous System of volatile anti-inflammatory agents.

Corbett, et al, “Drug repositioning for Alzheimer's Disease”, Nat Rev Drug Discov. 11, 833-846 (2012), reports that there is good in vitro evidence for NSAIDs having an effect on reducing amyloid accumulation, but conflicting clinical data do not provide strong enough rationale for benefits. And a review based on 15 studies concluded that NSAIDs could not be recommended for the treatment of Alzheimer's disease.

Rubio-Perez, et al, “A Review: Inflammatory Process in Alzheimer's Disease, Role of Cytokines”, The Scientific World Journal. 1-15 (2012), reports that it has been observed in epidemiological studies that treatment with NSAIDs decreases the risk for developing Alzheimer's Disease, but unfortunately, clinical trials of NSAIDs in Alzheimer's Disease patients using conventional oral administration have not been very fruitful.

Therefore, there is a need for a novel treatment for neurological conditions, such as, pain and neurodegenerative disorders, such as Alzheimer's disease.

### SUMMARY TO THE INVENTION

The present invention envisages the administration of a volatile form of an NSAID, or a derivative thereof, in vaporous form, for the treatment of a neurological disorder. The invention is advantageous because, inter alia, delivery of a therapeutically active agent via the olfactory tract provides a sufficient dose direct to the brain without exposing the patient to systemic doses of the active agent.

Thus, according to a first aspect of the invention there is provided a volatile form of an NSAID, or a derivative thereof, for use in the treatment of a neurological disorder.

The volatile form of an NSAID, or a derivative thereof, may comprise an NSAID, or a derivative thereof, which is inherently volatile. However, it is within the scope of the present invention that the NSAID which is therapeutically effective in the treatment of a neurological disorder may not be volatile per se, but may be presented in a form that is volatile.

Thus, the present invention particularly provides a volatile form of an NSAID, or a derivative thereof, for delivery via the olfactory tract of a patient; and for use in the treatment of a neurological disorder.

In another embodiment, the NSAID, or a derivative thereof, may be in the form of a solid solution with a second agent. It will be understood by a person skilled in the art that a solid solution is generally a mixture of two compounds. A solid solution is distinct from a compound per se. In a solid solution the crystal structure of the solvent (second agent) remains unchanged by addition of the solute (NSAID) and the mixture remains in a single homogeneous phase. One example of a solid solution is the crystallisation of salts from their liquid mixture.

It will be further understood by the person skilled in the art that more than one active agent, having the requisite properties, may be employed if warranted by biological or clinical findings.

Volatile forms of NSAIDs, whether substantially pure NSAIDs, solid solutions, etc., will generally be known to the person skilled in the art. A volatile form of an NSAID, or a derivative thereof, as hereinbefore described should be construed as a form of an NSAID, or a derivative thereof, which is capable of being vaporised (at atmospheric pressure). The temperature of vaporisation will vary depending upon, inter alia, the nature of the NSAID, for example, NSAIDs that vaporise at a temperature of ≦150° C. are especially suitable. However, it will be understood by the person skilled in the art that NSAIDs which vaporise at a temperature of >150° C. may still be used with an appropriate vaporisation means, e.g. a heating element.

The volatile form of the NSAID, or a derivative thereof, may desirably have a melting point (at atmospheric pressure) of ≦145° C., preferably ≦145° C., more preferably ≦140° C.

Preferably the volatile form of the NSAID, or a derivative thereof, does not degrade when heated to beyond its melting temperature and/or exhibits substantial sublimation.

A wide variety of NSAIDs, or a derivative thereof, may be used in the volatile form of the NSAID. As hereinbefore described, such forms include, but shall not be limited to solid solutions, and the like. Specific NSAIDs which can be mentioned include, but shall not be limited to, acetylsalicylic acid (aspirin), celecoxib, diclofenac, etodolac, fenoprofen, flurbiprofen, ibuprofen, indomethacin, ketoprofen, ketorolac, loxoprofen, methyl salicylate, nabumetone, naproxen, oxaprozin, piroxicam, salicylic acid, salsalate, sulindac and tolmetin, and derivatives thereof; and anti-inflammatory alkaloids, such as nicotine. Certain of the aforementioned NSAIDs are chiral, i.e. they possess at least one asymmetric carbon. It will be understood by the person skilled in the art that the NSAIDs according to the present invention may be provided as the individual enantiomers, as a non-racemic mixture, i.e. a mixture in which there is an enantiomeric excess of at least one enantiomer, or as a racemic mixture.

Certain NSAIDs which have a melting point (at atmospheric pressure) of ≦150° C., and which may be used as a therapeutically effective NSAID form, without the need for a solid solution, etc. Such NSAIDs include, but shall not be limited to, one or more of acetylsalicylic acid (aspirin); etodolac; fenoprofen; flurbiprofen; ibuprofen; ketoprofen; nabumetone; methyl salicylate, salicylic acid and salsalate; and derivatives thereof; and anti-inflammatory alkaloids, such as nicotine.

A preferred group of NSAIDs which may be mentioned is the group consisting of acetylsalicylic acid (aspirin); etodolac; fenoprofen; flurbiprofen; ibuprofen; ketoprofen; nabumetone; methyl salicylate, salicylic acid and salsalate; and derivatives thereof.

A more preferred group of NSAIDs which may be mentioned is the group consisting of ibuprofen; nabumetone; methyl salicylate and salicylic acid; and derivatives thereof.

Thus, according one aspect of the invention there is provided ibuprofen, and derivatives thereof, for use in the treatment of a neurological disorder.

According to another aspect of the invention there is provided nabumetone, and derivatives thereof, for use in the treatment of a neurological disorder.

According to another aspect of the invention there is provided methyl salicylate, and derivatives thereof, for use in the treatment of a neurological disorder.

According to another aspect of the invention there is provided salicylic acid, and derivatives thereof, for use in the treatment of a neurological disorder.

According to another aspect of the invention there is provided an anti-inflammatory alkaloid, such as nicotine, for use in the treatment of a neurological disorder.

One NSAID of particular interest is nabumetone, and derivatives thereof, which has a melting point of about 78° C., does not degrade when heated to beyond its melting temperature and exhibits substantial sublimation, even in moderately warm conditions. Additionally, the hydrophobic nature of nabumetone, and derivatives thereof, leads to good permeation across biological barriers. Nabumetone is a pro-drug, it is metabolised to the active form, 6-methoxy 2-naphthyl acetic acid (6-MNA), by enzymes such as Cytochrome P450 1A2 (CYP450 1A2), which are present in most regions of the brain. Another NSAID which may be mentioned that exhibits substantial sublimation is ibuprofen.

It is intended that the term neurological disorder shall include pain and neurodegenerative disorders, including neuroinflammatory disorders.

Pain may be associated with various diseases, disorders, or conditions, and may include various aetiologies. Illustrative types of pain treatable according to the present invention include cancer pain, postoperative pain, GI tract pain, spinal cord injury pain, visceral hyperalgesia, thalamic pain, headache (including stress headache and migraine), low back pain, neck pain, musculoskeletal pain, peripheral neuropathic pain, central neuropathic pain, neurodegenerative disorder related pain, and menstrual pain.

Neurodegenerative disorders may include one or more of Alzheimer's disease, memory loss, attention deficit symptoms associated with Alzheimer's disease, diffuse Lewy body type Alzheimer's disease, disorders associated with cognitive impairment, such as, but not limited to, mild cognitive impairment, Hereditary Cerebral Haemorrhage with Amyloidosis of the Dutch-Type, β-amyloid angiopathy and cerebral bleeding such as cerebral bleeding due to solitary cerebral amyloid angiopathy, vascular cognitive impairment, cerebrovascular dysfunction, prion infections, degenerative dementias, including dementias of mixed vascular and degenerative origin, frontotemporal dementia, pre-senile dementia, senile dementia, AIDS associated dementia, Parkinsonian disorders such as Parkinson's disease (PD), sub-acute sclerosing panencephalis, postencephalitic parkinsonism, dementia pugilistica, guam Parkinsonism-dementia complex, Pick's disease, multiple system atrophy (MSA), progressive supranuclear palsy (PSP), and corticobasal degeneration (CBD), Lewy body disease, Huntington's Disease, amyotrophic lateral sclerosis (ALS), multiple sclerosis, neurotraumatic diseases, acute stroke, epilepsy, mood disorders such as depression, schizophrenia and bipolar disorders, promotion of functional recovery post stroke, brain injury, especially traumatic brain injury, stroke and ischemia, neurosarcoidosis, anoxic brain damage, CNS vasculitis, glioma, Friedreich's Ataxia, Down Syndrome, cognitive loss, pain sensation and CNS trauma. In a preferred embodiment the neurodegenerative disorder is selected from one or more of Alzheimer's disease, Parkinsonism, dementia and Traumatic Brain Injury.

According to one aspect of the invention there is provided a volatile form of an NSAID, or a derivative thereof, for use in the treatment of a neurological disorder, wherein the neurological disorder comprises pain as hereinbefore defined.

According to another aspect of the invention there is provided a volatile form of an NSAID, or a derivative thereof, for use in the treatment of a neurological disorder, wherein the neurological disorder comprises a neurodegenerative disorder as hereinbefore defined.

Thus, in an especially preferred aspect of the present invention there is provided nabumetone, and derivatives thereof, in a volatile form, for the treatment of a neurological disorder. Thus, according to one aspect of the invention there is provided nabumetone, and derivatives thereof, in a volatile form for the treatment of pain. There is especially provided nabumetone, and derivatives thereof, in a volatile form for the treatment of a neurodegenerative disorder, such as, Alzheimer's disease.

It will be understood by the person skilled in the art that the volatile form of an NSAID, and derivatives thereof, may provide treatment and/or prophylaxis of a neurological disorder.

According to a yet further aspect of the invention there is provided a pharmaceutical composition comprising a volatile form of an NSAID, and derivatives thereof, as hereinbefore described, in association with a pharmaceutically acceptable adjuvant, diluent or carrier, for use in the treatment of a neurological disorder.

In the aforementioned pharmaceutical composition the NSAID, and derivatives thereof, may desirably be in solid form, preferably a finely divided particulate form. The finely divided particulate form of the NSAID, and derivatives thereof, will generally have a mass median diameter in the range of from about 0.01 to about 10 microns, e.g. having a mass median diameter of less than about 4 microns and especially of less than about 3 microns and more preferably of less than about 2.8 microns. Desirably not more than about 5% by weight of the particles to have a diameter of greater than about 10 microns, and more preferably not less than about 90% by weight of the particles to have a diameter of less than about 6 microns. The finely divided NSAID, and derivatives thereof, may be prepared in the desired particle size range use techniques known to the person skilled in the art, for example, using a ball mill, a fluid energy mill, by precipitation or by spray drying. The carrier may be prepared by spray drying or grinding and subsequently separating out the desired fraction, for example by air classification and/or sieving.

In a particular embodiment of the invention the pharmaceutical composition as hereinbefore described may comprise a volatile form of an NSAID, and derivatives thereof, in association with a propellant, i.e. for use in the treatment of a neurological disorder. It should be understood the pharmaceutical composition according to this aspect of the invention will generally comprises a true solution of an NSAID, and derivatives thereof, in the propellant. NSAIDs, such as nabumatone, are generally hydrophobic and soluble in non-polar solvents, including inter alia, in liquefied propellants.

According to this embodiment the pharmaceutical composition as hereinbefore described may comprise the NSAID, and derivatives thereof, dissolved in a propellant, optionally with additional materials such as an anti-flocculent, a solubiliser or an agent that enhances permeability across the mucosal barrier. Such components will be familiar to those skilled in the art of preparing stable suspensions or dispersions and would preferably be volatile. For delivery of therapeutically active agents via the olfactory tract, a particularly suitable excipient in the composition of the present invention may be a fragrance. However, it will be understood by the person skilled in the art that preferably the NSAID should be administered in a substantially pure form, i.e. without the use of the aforementioned additional materials, such as a mucosal permeability enhancer.

When the pharmaceutical composition comprises the NSAID, and derivatives thereof, dissolved in a propellant, any conventionally known pharmaceutically acceptable propellants may be used in the composition of the invention. The pharmaceutical composition of this aspect of the invention may be, for example, presented in a pressurised metered dose inhaler (pMDI), which will typically comprise a canister including a metered dose valve mounted on an aerosol container filled medicinal inhalation formulation and an actuator including a nasal- or mouthpiece. Such aerosol formulations used in a canister for pMDIs may typically be prepared by dissolving or dispersing, via particles of the NSAID, and derivatives thereof, in one or more liquefied propellants, e.g. CFC propellants or non-CFC propellants, such as a hydrofluoroalkane propellant, e.g. 1,1,1,2-tetrafluoroethane (HFA134a) and/or 1,1,1,2,3,3,3-heptafluoropropane (HFA227). If desired and/or deemed necessary, the formulation may comprise other components, such as excipients, co-solvents, and suspending aids.

Therefore, according to this aspect of the invention the pharmaceutical composition may comprise an NSAID, and derivatives thereof, as an HFA solution, e.g. as a solution of HFA134a or HFA227.

Yet another embodiment comprises the NSAID, and derivatives thereof, dissolved or dispersed in a propellant such as a hydrofluoroalkane (HFA) having suitable vapour pressure such that it is liquid at high atmospheric pressure but rendered volatile when expelled from the device on encountering atmospheric pressure. On actuation of a valve associated with the reservoir a dose of the NSAID, and derivatives thereof, is delivered via inspiration through the nasal orifices of a patient.

In addition, the propellant may comprise other non-flammable propellants, such as carbon dioxide.

When the composition of the present invention includes a fragrance, the fragrance will preferably be in the form of an oil, e.g. a volatile oil. A wide variety of such fragrance oils are known to the person skilled in the art, and include fragrance oils that provide relief for nasal passages, such as, peppermint oil, eucalyptus oil, spearmint oil, camphor oil, rosemary oil and the like.

In another embodiment of the invention the pharmaceutical composition as hereinbefore described may comprise a volatile form of an NSAID, and derivatives thereof, in association with a solvent, i.e. for use in the treatment of a neurological disorder.

Suitable organic solvents include pharmaceutically acceptable solvents, for example, aliphatic, cycloaliphatic and aromatic hydrocarbons (pentane, hexane, petroleum ether, cyclohexane, methyl cyclohexane, benzene, toluene, xylene), aliphatic halogenated hydrocarbons (methylene chloride, chloroform, di- and tetra-chloroethane), nitriles (acetonitrile, propionitrile, benzonitrile), ethers (diethyl ether, dibutyl ether, t-butylmethyl ether, ethylene glycol dimethyl ether, ethylene glycol diethyl ether, diethylene glycol dimethyl ether, tetrahydrofuran, dioxane, diethylene glycol monomethyl or monoethyl ether), ketones (acetone, methyl isobutyl ketone), carboxylic acid esters and lactones (ethyl or methyl acetate, valerolactone), N-substituted lactams (N-methylpyrrolidone), carboxamides (dimethylamide, dimethylformamide), acyclic ureas (dimethyl imidazoline) and sulfoxides and sulfones (dimethyl sulfoxide, dimethyl sulfone, tetramethylene sulfoxide, tetramethylene sulfone) and alcohols (methanol, ethanol, propanol, butanol, ethylene glycol monomethyl ether, ethylene glycol monoethyl ether, diethylene glycol monomethyl ether). The organic solvents may be used alone or in a mixture of at least two solvents.

The organic solvent may comprise a wetting agent, for example, ionic or non-ionic wetting agents commonly used in the formulation of pharmaceuticals, including but not limited to castor oil derivatives, cholesterol, polyglycolyzed glycerides, acetylated monoglycerides, sorbitan fatty acid esters, poloxamers, polysorbates, polyoxyethylene sorbitan fatty acid esters, polyoxyethylene compounds, monoglycerides or ethoxylated derivatives thereof, diglycerides or polyoxyethylene derivatives thereof, sodium docusate, sodium laurylsulfate, cholic acid or derivatives thereof, ethoxylated alcohols, ethoxylated esters, ethoxylated amides, polyoxypropylene compounds, propoxylated alcohols, ethoxylated/propoxylated block polymers, propoxylated esters, alkanolamides, amine oxides, fatty acid esters of polyhydric alcohols, ethylene glycol esters, diethylene glycol esters, propylene glycol esters, glycerol esters, polyglycerol fatty acid esters, SPAN's (e.g., sorbitan esters), TWEEN's (i.e., sucrose esters), glucose (dextrose) esters, alkali metal sulfates, quaternary ammonium compounds, amidoamines, and aminimides, simethicone, lecithins, alcohols, phospholipids, and mixtures thereof. A preferred organic solvent wetting agent is a polyhydroxy alcohol, such as propylene glycol.

The pharmaceutical compositions of the invention may include a liquid nasal carrier. As used herein, the term “liquid nasal carrier” includes a solution, emulsion, or suspension designed for delivery of the NSAID, and derivatives thereof, to the nasal mucosa. The liquid nasal carrier includes a diluent suitable for application to the nasal mucosa. Suitable diluents include aqueous or non-aqueous diluents or combination thereof, depending upon, inter alia, the nature of the NSAID. Examples of aqueous diluents include, but are not limited to, saline, water, dextrose or combinations thereof. Non-aqueous diluents include, but are not limited to, alcohols, particularly polyhydroxy alcohols such as propylene glycol, polyethylene glycol, glycerol, and vegetable and mineral oils.

The pharmaceutical composition of the invention may additionally comprise one or more of a dispersant, anti-flocculants and solubiliser, which would be known to the person skilled in the art.

Some pharmaceutical formulations comprising a volatile form of an NSAID, and derivatives thereof, are novel per se.

It will be understood that in order to liquefy and/or volatilise the form of an NSAID, and derivatives thereof, it may be desirable to provide an energy-generating component. Thus, one such novel pharmaceutical formulation comprises a volatile form of an NSAID, and derivatives thereof, including an energy-generating component.

The energy-generating component may comprise, for example, one or more of, a heat source and an ultrasound source (sonicator).

In one embodiment of the invention the energy-generating component may comprise a heat source, for example, one or more heating elements, suitable for vaporising the NSAID, and derivatives thereof. Optionally, the heat energy may be transferred to the NSAID, and derivatives thereof, by means of electromagnetic waves, in particular radiant heat or microwaves. Thus, for example, the energy-generating component may comprise a personal vaporiser or “e-cigarette”, i.e. components include a power source, a liquid container system and an atomiser. Alternatively, the liquid container system and atomiser may be combined in a cartomiser.

In another embodiment of the invention the energy source may comprise a chamber, surrounded by or containing an energy-generating device, wherein the energy-generating device comprises a sonicator. A dosage form comprising the volatile form of an NSAID, and derivatives thereof, as hereinbefore described, or a composition thereof, may be introduced into the sonicator chamber. Upon activation of the sonicator, the NSAID, and derivatives thereof, will be vaporised such that the NSAID, and derivatives thereof, is generally propelled into the nasal cavity of a patient.

According to a further aspect of the invention there is provided a method of treatment of a neurological disorder which comprises administering to a mammal a therapeutically effective amount of a volatile form of an NSAID, or a derivative thereof, in vapour form.

According to this aspect of the invention there is provided a method of treatment as hereinbefore described, wherein the neurological disorder comprises pain as hereinbefore defined.

According to another aspect of the invention there is provided a method of treatment as hereinbefore described, wherein the neurological disorder comprises a neurodegenerative disorder as hereinbefore defined.

Thus, in an especially preferred aspect of the present invention there is provided a method of treatment of a neurological disorder which comprises the administration of nabumetone, and derivatives thereof, in a volatile form. Thus, according to another aspect of the invention there is provided a method of treatment of pain which comprises the administration of nabumetone, and derivatives thereof, in a volatile form. There is especially provided a method of treatment of a neurodegenerative disorder, such as, Alzheimer's disease, which comprises the administration of nabumetone, and derivatives thereof, in a volatile form.

Generally, in the method according to this aspect of the invention, a “therapeutically effective amount” of the NSAID form of the present invention or a pharmaceutical composition of the present invention will depend upon, inter alia, the relative efficacy of the chosen NSAID, the severity of the disorder being treated, the weight of the patient, etc. However, the NSAID, and derivatives thereof, will typically be administered once or more times a day for example 1, 2, 3 or 4 times daily, with typical total daily doses in the range of from about 0.0001 to about 5 mg/kg/day; or from about 0.005 to about 5 mg/kg/day; or from about 0.05 to about 5 mg/kg/day.

As used herein, the term “pharmaceutically acceptable adjuvant, diluent or carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavouring agents, dyes, such like materials and combinations thereof, as would be known to one of ordinary skill in the art (see, for example, Remington's Pharmaceutical Sciences, 18^{th} Ed. Mack Printing Company, 1990, pp. 1289-1329). Except insofar as any conventional carrier is incompatible with the active ingredient, its use in the therapeutic or pharmaceutical compositions is contemplated.

It will be understood by the person skilled in the art that the term “derivative” shall include, inter alia, pharmaceutically acceptable salts, solvates, esters and pro-drugs. Non-pharmaceutically acceptable salts, solvates, esters and pro-drugs should also be considered to fall within the scope of the present invention, since those may be useful in the preparation of pharmaceutically acceptable salts, solvates or pro-drugs.

The preparation of salts, solvates and pro-drugs can be carried out by methods known to the person skilled in the art. It will be appreciated that non-pharmaceutically acceptable salts, solvates or pro-drugs also fall within the scope of the invention since those may be useful in the preparation of pharmaceutically acceptable salts, solvates or pro-drugs. Methods of solvation are generally known within the art. Suitable solvates are pharmaceutically acceptable solvates.

As used herein, the term “salt” shall generally mean a “pharmaceutically acceptable salt” and refers to salts that retain the biological effectiveness and properties of the compounds of this invention and, which are not biologically or otherwise undesirable. Similarly, the “ester” shall mean a “pharmaceutically acceptable ester”.

Salts are desirably pharmaceutically acceptable base addition salts, which can be formed with inorganic and organic bases. Inorganic bases from which salts can be derived include, for example, sodium, potassium, lithium, ammonium, calcium, magnesium, iron, zinc, copper, manganese, aluminium, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine Generally, such salts can be prepared by reacting free acid forms of an NSAID with a stoichiometric amount of the appropriate base (such as Na, Ca, Mg, or K hydroxide, carbonate, bicarbonate, or the like), or by reacting free base forms of an NSAID with a stoichiometric amount of an appropriate acid. Such reactions are typically carried out in water or in an organic solvent, or in a mixture of the two. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Other salts include ammonium or amino acid salts which are water soluble thereby being preferred. Complex salts with basic amino acids can be used directly and mixed salts with neutral or acidic amino acids are previously converted into the alkali metal, alkaline earth metal or ammonium salts. A preferred amino acid is lysine. Other methods also known for medicaments, in which the active material is adsorbed onto aluminium oxide gels, can also be carried out with the NSAIDs according to the present invention. The NSAID salts produced can then be further worked up in the manner described hereinbefore. Preferably, the NSAID salts are prepared indirectly by adding the bases required for the salt formation. Amino acid salts may comprise an essential amino acid, such as, histidine, isoleucine, leucine, lysine, methionine, phenylalanine, threonine, tryptophan, valine and tyrosine; or a non-essential amino acid, such as, alanine, arginine, aspartate, cysteine, glutamate, glutamine, glycine, proline, serine, asparagines and selenocysteine. Alternatively, the salt may comprise an amino sugar, such as meglumine.

Esters will generally be pharmaceutically acceptable esters which may be produced by reacting the propanoic acid of the NSAID with an appropriate alcohol. Esters of pharmaceutically acceptable alcohols can be formed with organic alcohols, such esters include, but shall not be limited to, e.g. acetate, acetoxyethyl ester (axetil), aspartate, benzoate, besylate, camsylate, cinnamate, citrate, edisylate, esylate, ethanesulfonate, formate, fumarate, gluceptate, gluconate, glucuronate, glycolate, hexafluorophosphate, hibenzate, isethionate, lactate, malate, maleate, malonate, mandelate, mesylate, methanesulfonate, methylsulfate, 2-napsylate, naphthylate, nicotinate, orotate, oxalate, palmitate, pamoate, propionate, pyruvate, saccharate, salicylate, stearate, succinate, tartrate, p-toluenesulfonate, tosylate, trifluoroacetate, and the like.

However, since formation of a salt or ester of an NSAID will generally increase the melting point/vaporisation point of the active ingredient, preferably the NSAID will be present in substantially free form.

The therapeutically effective NSAID, and derivatives thereof, of the present invention may be in crystalline form either as free compounds or as solvates (e.g. hydrates) and it is intended that both forms are within the scope of the present invention. Methods of solvation are generally known within the art. Suitable solvates are pharmaceutically acceptable solvates. In a particular embodiment the solvate is a hydrate.

The present invention also provides pro-drugs of the NSAIDS, and derivatives thereof, of the present invention of the present invention that converts in vivo to the compounds of the present invention. A pro-drug is an active or inactive compound that is modified chemically through in vivo physiological action, such as hydrolysis, metabolism and the like, into a compound of this invention following administration of the pro-drug to a subject. The suitability and techniques involved in making and using pro-drugs are well known by those skilled in the art. Pro-drugs can be conceptually divided into two non-exclusive categories, bioprecursor pro-drugs and carrier pro-drugs. See The Practice of Medicinal Chemistry, Ch. 31-32 (Ed. Wermuth, Academic Press, San Diego, Calif., 2001). Generally, bioprecursor pro-drugs are compounds, which are inactive or have low activity compared to the corresponding active drug compound that contain one or more protective groups and are converted to an active form by metabolism or solvolysis. Both the active drug form and any released metabolic products should have acceptably low toxicity.

When used in the manufacture of a pharmaceutical composition of the invention, excipients e.g. an adjuvant, diluent or carrier, may be present in an amount ranging from about 0.1% to about 2% or from about 2% to about 70% or 85% by weight (w/w or w/v), including 0.15%, about 0.2%, about 0.5%, about 1%, about 1.5%, about 5%, about 10%, about 15%, about 20%, about 30%, about 40%, about 45%, about 48%, about 85%, about 78%, and further including about 0.25%, about 0.35%, about 0.5%, about 1.0%, about 2.0%, about 2.5%, about 7.5%, about 13%, about 14%, about 16%, about 17%, about 17.5%, about 18%, about 25%, about 25.5%, about 27%, about 27.5%, about 32%, about 50%, about 53%, about 60%, about 62%, about 70%, about 75%, about 80%, and so on.

According to a further aspect of the invention there is provided a process for the manufacture of a pharmaceutical composition as hereinbefore described which comprises admixing a volatile form of an NSAID, and derivatives thereof, with a pharmaceutically acceptable adjuvant, diluent or carrier.

According to a yet further aspect of the invention there is provided a kit for delivery of a volatile form of an NSAID, and derivatives thereof, for use in the treatment of a neurological disorder, said kit comprising:
a volatile form of an NSAID, and derivatives thereof, or a pharmaceutical composition thereof; and
an energy-generating component.

According to a further aspect of the invention the energy-generating component comprises a personal vaporiser or “e-cigarette”.

1. A volatile form of an NSAID, and derivatives thereof, for use in the treatment of a neurological disorder.

2. A volatile form of an NSAID according to claim 1 wherein the NSAID, and derivatives thereof, is inherently volatile.

3. A volatile form of an NSAID according to claim 1 wherein the NSAID, and derivatives thereof, is not inherently volatile, but is presented in a form that is volatile.

4. A volatile form of an NSAID according to claim 1 wherein the NSAID, and derivatives thereof, is in the form of a solid solution with a second agent.

5. A volatile form of an NSAID according to claim 4 wherein the second agent is a therapeutically active agent.

6. A volatile form of an NSAID according to claim 4 wherein the second agent is therapeutically inert.

7. A volatile form of an NSAID according to claim 3 wherein the NSAID vaporises at a temperature of ≦150° C.

8. A volatile form of an NSAID according to any one of the preceding claims wherein the NSAID is selected from the group comprising acetylsalicylic acid (aspirin), celecoxib, diclofenac, etodolac, fenoprofen, flurbiprofen, ibuprofen, indomethacin, ketoprofen, ketorolac, loxoprofen, methyl salicylate, nabumetone, naproxen, oxaprozin, piroxicam, salicylic acid, salsalate, sulindac and tolmetin, and derivatives thereof, and anti-inflammatory alkaloids, such as nicotine.

9. A volatile form of an NSAID according to any one of the preceding claims wherein the NSAID is selected from the group comprising acetylsalicylic acid (aspirin), celecoxib, diclofenac, etodolac, fenoprofen, flurbiprofen, ibuprofen, indomethacin, ketoprofen, ketorolac, loxoprofen, methyl salicylate, nabumetone, naproxen, oxaprozin, piroxicam, salicylic acid, salsalate, sulindac and tolmetin, and derivatives thereof.

10. A volatile form of an NSAID according to claim 8 or 9 wherein the NSAID is selected from the group comprising acetylsalicylic acid (aspirin); etodolac; fenoprofen; flurbiprofen; ibuprofen; ketoprofen; nabumetone; methyl salicylate and salicylic acid, salsalate; and derivatives thereof.

11. A volatile form of an NSAID according to claim 10 wherein the NSAID is selected from the group comprising ibuprofen; nabumetone; methyl salicylate and salicylic acid; and derivatives thereof.

12. A volatile form of an NSAID according to claim 11 wherein the NSAID is ibuprofen, and derivatives thereof.

13. A volatile form of an NSAID according to claim 12 wherein the NSAID is nabumetone, and derivatives thereof.

14. A volatile form of an NSAID according to claim 13 wherein the NSAID is methyl salicylate, and derivatives thereof.

15. A volatile form of an NSAID according to claim 14 wherein the NSAID is salicylic acid, and derivatives thereof.

16. A volatile form of an NSAID according to claim 8 wherein the NSAID is an anti-inflammatory alkaloid, such as nicotine.

17. A volatile form of an NSAID according to any one of the preceding claims wherein the neurological disorder comprises pain.

18. A volatile form of an NSAID according to any one of claims 1 to 16 wherein the neurological disorder comprises a neurodegenerative disorder.

19. A volatile form of an NSAID according to claim 18 wherein the neurological disorder is a neuroinflammatory disorder.

20. A volatile form of an NSAID according to claim 18 wherein the neurodegenerative disorder is selected from one or more of Alzheimer's disease, Parkinsonism, dementia and Traumatic Brain Injury.

21. A volatile form of an NSAID according to claim 17 wherein the NSAID is nabumetone, and derivatives thereof, for the treatment of pain.

22. A volatile form of an NSAID according to claim 18 wherein the NSAID is nabumetone, and derivatives thereof, for the treatment of a neurodegenerative disorder.

23. A volatile form of an NSAID according to claim 22 wherein the NSAID is nabumetone, and derivatives thereof, for the treatment of Alzheimer's disease.

24. A volatile form of an NSAID according to any one of the preceding claims wherein the treatment comprises prophylaxis of a neurological disorder.

25. A pharmaceutical composition comprising a volatile form of an NSAID, and derivatives thereof, in association with a pharmaceutically acceptable adjuvant, diluent or carrier, for use in the treatment of a neurological disorder.

26. A pharmaceutical composition comprising according to claim 25 wherein the NSAID, and derivatives thereof, is in solid, finely divided particulate form.

27. A pharmaceutical composition according to claim 26 wherein the particles of the NSAID, and derivatives thereof, have a mass median diameter in the range of from about 0.01 to about 10 microns.

28. A pharmaceutical composition according to claim 26 or 27 wherein not more than 5% by weight of the NSAID particles have a diameter of greater than 10 microns.

29. A pharmaceutical composition according to any one of claims 25 to 28 wherein the composition comprises a propellant.

30. A pharmaceutical composition according to claim 29 wherein the propellant is a CFC propellant or a non-CFC propellant, and mixtures thereof.

31. A pharmaceutical composition according to claim 30 wherein the non-CFC propellant is a hydrofluoroalkane.

32. A pharmaceutical composition according to claim 31 wherein the hydrofluoroalkane is 1,1,1,2-tetrafluoroethane (HFA134a) and/or 1,1,1,2,3,3,3-heptafluoropropane (HFA227).

33. A pharmaceutical composition according to claim 29 wherein the propellant comprises carbon dioxide.

34. A pharmaceutical composition according to any one of claims 25 to 33 wherein the composition includes a fragrance.

35. A pharmaceutical composition according to any one of claims 25 to 34 wherein the composition comprises a volatile form of an NSAID, and derivatives thereof, in association with a solvent.

36. A pharmaceutical composition according to claim 35 wherein the composition includes a pharmaceutically acceptable solvent is an organic solvent.

37. A pharmaceutical composition according to claim 36 wherein the organic solvent is a wetting agent.

38. A pharmaceutical composition according to claim 37 wherein the wetting agent is a polyhydroxy alcohol.

39. A pharmaceutical formulation comprising a volatile form of an NSAID, and derivatives thereof, including an energy-generating component.

40. A pharmaceutical formulation according to claim 39 wherein the energy-generating component is selected from one or more of a heat source and an ultrasound source.

41. A pharmaceutical formulation according to claim 40 wherein the heat source comprises one or more heating elements.

42. A pharmaceutical formulation according to claim 40 wherein the energy-generating device comprises a sonicator.

43. A method of treatment of a neurological disorder which comprises administering to a mammal a therapeutically effective amount of a volatile form of an NSAID, or a derivative thereof, in vapour form.

44. A method according to claim 43 wherein the NSAID, and derivatives thereof, is inherently volatile.

45. A method according to claim 43 wherein the NSAID, and derivatives thereof, is not inherently volatile, but is presented in a form that is volatile.

46. A method according to claim 43 wherein the NSAID, and derivatives thereof, is in the form of a solid solution with a second agent.

47. A method according to claim 46 wherein the second agent is a therapeutically active agent.

48. A method according to claim 46 wherein the second agent is therapeutically inert.

49. A method according to any one of claims 43 to 48 wherein the NSAID is selected from the group comprising acetylsalicylic acid (aspirin), celecoxib, diclofenac, etodolac, fenoprofen, flurbiprofen, ibuprofen, indomethacin, ketoprofen, ketorolac, loxoprofen, methyl salicylate, nabumetone, naproxen, oxaprozin, piroxicam, salicylic acid, salsalate, sulindac and tolmetin, and derivatives thereof, and anti-inflammatory alkaloids, such as nicotine.

50. A method according to any one of claims 43 to 49 wherein the NSAID is selected from the group comprising acetylsalicylic acid (aspirin), celecoxib, diclofenac, etodolac, fenoprofen, flurbiprofen, ibuprofen, indomethacin, ketoprofen, ketorolac, loxoprofen, methyl salicylate, nabumetone, naproxen, oxaprozin, piroxicam, salicylic acid, salsalate, sulindac and tolmetin, and derivatives thereof.

51. A method according to claim 49 or 50 wherein the NSAID is selected from the group comprising acetylsalicylic acid (aspirin); etodolac; fenoprofen; flurbiprofen; ibuprofen; ketoprofen; nabumetone; methyl salicylate, salicylic acid and salsalate; and derivatives thereof.

52. A method according to claim 51 wherein the NSAID is selected from the group comprising ibuprofen; nabumetone; methyl salicylate and salicylic acid; and derivatives thereof.

53. A method according to claim 51 wherein the NSAID is ibuprofen, and derivatives thereof.

54. A method according to claim 51 wherein the NSAID is nabumetone, and derivatives thereof.

55. A method according to claim 51 wherein the NSAID is methyl salicylate, and derivatives thereof.

56. A method according to claim 51 wherein the NSAID is salicylic acid, and derivatives thereof.

56. A method according to claim 49 wherein the NSAID is anti-inflammatory alkaloids, such as nicotine.

57. A method according to any one of claim 43 wherein the neurological disorder comprises pain.

58. A method according to any one of claims 43 to 56 wherein the neurological disorder comprises a neurodegenerative disorder.

59. A method according to claim 58 wherein the neurological disorder is a neuroinflammatory disorder.

60. A method according to claim 58 wherein the neurological disorder is a neurodegenerative disorder is selected from one or more of Alzheimer's disease, Parkinsonism, dementia and Traumatic Brain Injury.

61. A method according to claim to claim 57 wherein the NSAID is nabumetone, and derivatives thereof, for the treatment of pain.

62. A method according to claim to claim 58 wherein the NSAID is nabumetone, and derivatives thereof, for the treatment of a neurodegenerative disorder.

63. A method according to claim 62 wherein the NSAID is nabumetone, and derivatives thereof, for the treatment of Alzheimer's disease.

64. A method according to any one of claims 43 to 63 wherein the treatment comprises prophylaxis of a neurological disorder.

65. A method according to any one of claims 43 to 64 wherein the total daily dose of the NSAID, and derivatives thereof, is in the range of from about 0.0001 to about 5 mg/kg/day.

66. A process for the manufacture of a pharmaceutical composition according to any one of claims 25 to 42 which comprises admixing a volatile form of an NSAID, and derivatives thereof, with a pharmaceutically acceptable adjuvant, diluent or carrier.

67. A kit for delivery of a volatile form of an NSAID, and derivatives thereof, for use in the treatment of a neurological disorder, said kit comprising:
a volatile form of an NSAID, and derivatives thereof, or a pharmaceutical composition thereof; and
an energy-generating component.

68. A kit according to claim 67 wherein the energy-generating component comprises a personal vaporiser or “e-cigarette”,

69. A volatile form of an NSAID, composition, method, kit or process as hereinbefore described with reference to the accompanying description.
